(19) United States
(12) Patent Application Publication
(10) Pub. No.: US 2026/0240161 A1
(43) Pub. Date: Aug. 20, 2026

(54) Low Dose Fipronil Feed

(71) Applicant: Scimetrics Limited Corp, Wellington, CO (US)

(72) Inventors: David Matthew Poché, Wellington, CO (US); Richard Poché, Wellington, CO (US)

(73) Assignee: Scimetrics Limited Corp., Wellington, CO (US)

(21) Appl. No.: 19/645,936

(22) Filed: Apr. 13, 2026

Related U.S. Application Data
(63) Continuation-in-part of application No. 18/581,087, filed on Feb. 19, 2024.
(60) Provisional application No. 63/446,601, filed on Feb. 17, 2023.

(51) Int. Cl. A01N 47/02; A01N 25/00; A01N 43/56; A01P 7/02

(52) U.S. Cl. CPC A01N 47/02; A01N 25/002; A01N 43/56; A01P 7/02

(57) ABSTRACT

A vector control composition and corresponding methods of use, the vector control composition including an active agent effective to decrease the fitness of a vector capable of transmitting a pathogen, whereby the active agent includes an acaricide or a low dose of an acaricide, whereby the active agent can be formulated for oral delivery to a host of the vector, and whereby the host can be a rodent.

### I. BACKGROUND OF THE INVENTION

On a global scale, ticks are recognized as one of the main arthropod pathogen vectors of disease agents of humans and animals, and thus are of considerable medical importance. Ticks and wildlife species encompass vector-host relationships of increasing medical and veterinary concern, with many notable tick-borne diseases (such as anaplasmosis, babesiosis, ehrlichiosis, and Lyme disease) attracting substantial medical attention. Vector control is regarded as one of the more promising means for reducing human tick bites and preventing pathogen transmission. However, conventional methods, such as area-wide broadcast applications, present management concerns, including logistical and economic hurdles, the indiscriminate targeting of non-target organisms, such as pollinators, and the accelerated development of insecticidal resistance. Accordingly, there exists a need for additional, more discriminate methods of vector control; the present invention fulfills this need and provides further related advantages.

### II. SUMMARY OF THE INVENTION

A vector control composition and corresponding methods of use, the vector control composition including an active agent effective to decrease the fitness of a vector capable of transmitting a pathogen, whereby the active agent includes an acaricide or a low dose of an acaricide, whereby the active agent can be formulated for oral delivery to a host of the vector, and whereby the host can be a rodent.

### III. A BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates vector-host association, whereby tick larvae attach to a white-footed mouse host and begin blood feeding. Fully engorged, replete tick larvae drop from the host and begin nymph development. Infected nymphs can then bite humans.

FIG. 1B illustrates the impact of fipronil feed consumption by white-footed mouse hosts on development of blood-feeding tick larvae, whereby tick larvae blood feeding on mice that have consumed fipronil die and thus, are prevented from feeding to repletion and detaching, subsequently preventing nymph development and reducing the risk of human nymph bites.

FIG. 2 shows a table summarizing the amount of fipronil consumed by mice in treatment groups.

FIG. 3 shows a table summarizing tick larvae attachment status and feeding status for mice in treatment groups and corresponding control groups.

FIG. 4 shows a table summarizing tick larvae detached status and efficacy of fipronil in preventing tick larvae parasitizing mice from feeding to repletion post exposure to fipronil.

FIG. 5 shows a table summarizing tick larvae molting success and efficacy of fipronil in preventing tick larvae parasitizing mice from reaching the nymphal stage post exposure to fipronil.

FIG. 6 shows a table of concentrations of fipronil sulfone (a metabolite of fipronil) in plasma samples from mice in treatment groups.

FIG. 7 shows a table summarizing the amount of fipronil consumed by mice in treatment groups.

FIG. 8 shows a table summarizing tick larvae attachment status and feeding status for mice in treatment groups and corresponding control groups.

FIG. 9 shows a table summarizing tick larvae detached status and efficacy of fipronil in preventing tick larvae parasitizing mice from feeding to repletion post exposure to fipronil.

FIG. 10 shows a table summarizing tick larvae molting success and efficacy of fipronil in preventing tick larvae parasitizing mice from reaching the nymphal stage post exposure to fipronil.

FIG. 11 shows a table of concentrations of fipronil sulfone (a metabolite of fipronil) in plasma samples from mice in treatment groups.

### IV. DETAILED DESCRIPTION OF THE PREFERRED EMBODIMENTS

Disclosed herein are inventive compositions and methods of using the same to control a pathogen by controlling its vector via a vector control composition which includes a pesticide that targets the vector of the pathogen. The vector control composition can be delivered (or administered) to the vector, such as via a host of the vector, to decrease the fitness of the vector which correspondingly decreases the fitness of the pathogen to control the spread of vector-borne diseases in humans and animals.

### Pathogen Vector

The present vector control composition can target a vector comprising an acarine (an arachnid of the subclass Acari) which can transmit a pathogen from one organism to another. As to particular embodiments, the vector can be a parasitic acarine. As to particular embodiments, the vector can be a tick.

Numerous tick species are significant pests which can cause harm to both humans and animals (for example, but not limited to, wildlife, livestock, farm animals, zoo animals, companion animals, or the like). Ticks are obligate, blood-feeding ectoparasites that infest the skin of another organism (referred to herein as a “host”) and depend on their host for sustenance, maturation, and proliferation. If the tick is carrying a pathogen, it can transfer this pathogen into the bloodstream of a host during blood-feeding. In addition to transmitting pathogens and their associated diseases, ticks can also elicit a local reaction at the tick bite site, whereby the host's skin may become irritated, itchy, red, and/or swollen. Further, in some cases, the tick bite site may become infected.

The present vector control composition can be employed to control any susceptible tick. As illustrative, nonlimiting examples, the vector control composition may be especially useful for controlling Ixodes scapularis, also known as the blacklegged tick or deer tick; Amblyomma americanum, also known as the lone star tick; Haemaphysalis longicornis, also known as the Asian longhorned tick; and/or Rhipicephalus microplus, also known as the cattle fever tick; however, said again, the present vector control composition is not limited to controlling only these parasitic acarines.

### Pathogen

Ticks can act as vectors for various pathogens, whereby as used herein, the term “pathogen” refers to an infectious biological agent which can cause disease and/or disease symptoms in a human or animal by, for example, directly damaging host cells, tissues, and/or organs during the process of infection; producing toxins that cause damage to host cells, tissues, and/or organs; triggering an immune/inflammatory response which may contribute to host cell, tissue, and/or organ damage; interfering with host physiological and metabolic processes; inducing hypersensitivity reactions; or the like. Pathogens transmitted by ticks can include, but are not limited to, bacteria, viruses, parasites, and protozoa.

As illustrative, nonlimiting examples, ticks can transmit Borrelia burgdorferi, a causative agent of Lyme disease; Borrelia mayonii, a causative agent of Lyme disease; Anaplasma phagocytophilum, a causative agent of anaplasmosis; Babesia microti, a causative agent of babesiosis; Ehrlichia muris eauclairensis, Ehrlichia chaffeensis, and Ehrlichiosis ewingii, causative agents of ehrlichiosis; Borrelia miyamotoi, a causative agent of Borrelia miyamotoi disease; Powassan virus, a causative agent of Powassan virus disease; Heartland virus, a causative agent of Heartland virus disease; Bourbon virus, a causative agent of Bourbon virus disease; Francisella tularensis, a causative agent of tularemia; bacteria in the genus Rickettsia, causative agents of rickettsiosis; and the causative agent (unknown) of southern tick-associated rash illness (STARI). The present vector control composition may be useful for controlling any one or more of these pathogens and/or associated diseases.

### Host

The host of the vector which the present vector control composition can target may be a rodent, defined as a mammal characterized by a single pair of continuously growing incisors in each of the upper and lower jaws. The order Rodentia constitutes about 40% of all mammal species, categorized into five main suborders: Myomorpha (mouse-like rodents), Sciuromorpha (squirrel-like rodents), Hystricomorpha (porcupine-like rodents and cavy-like rodents), Castorimorpha (beaver-like rodents), and Anomaluromorpha (scaly-tailed squirrels and relatives).

As to particular embodiments, the host of the vector which the present vector control composition can target may be a mouse, belonging to the superfamily Muroidea. As to particular embodiments, the mouse can belong to the family Muridae (Old World mice) or the family Cricetidae (New World mice). Regarding the latter, as to particular embodiments, the mouse can belong to the Peromyscus genus (often collectively called “deer mice”). As to particular embodiments, the mouse can be a white-footed mouse (Peromyscus leucopus) which is a frequent host for immature I. scapularis and primary reservoir for Borrelia burgdorferi in the midwestern and northeastern United States where human Lyme disease incidence is prevalent.

Conventionally, broadcast acaricide application has been attempted to control parasitizing ticks; however, such a distribution of acaricides over a large, non-localized area can present logistical and economic hurdles, environmental concerns, and potential for insecticide resistance.

As an alternative approach, the present invention generally comprises orally delivering a pesticide to a host via a feed for the host which includes the pesticide to consequently deliver the pesticide to a vector which blood-feeds on the host, whereby the pesticide can be effective to decrease the fitness of the vector. As to particular embodiments, the present invention comprises orally delivering an active agent to a rodent via a feed for the rodent which includes the active agent to consequently deliver the active agent to a tick which blood-feeds on the rodent, whereby the active agent can be effective to decrease the fitness of the tick.

For such oral delivery to a host, such as a rodent, the present vector control composition can be formulated as a feed which contains an active agent effective to decrease the fitness of a tick capable of transmitting a pathogen.

### Vector Control Composition

As used herein, the term “vector control composition” refers to a composition including an active agent effective to control or decrease the fitness of a vector capable of transmitting a pathogen.

As used herein, the term “active agent” refers to a component of a composition which is responsible for and/or contributes significantly to the intended physiological effect or outcome following delivery of the composition.

As used herein “decreasing the fitness of a vector” refers to any disruption in the physiology of a vector and/or activity of a vector as a consequence of delivery of the vector control composition described herein, including but not limited to any one or more of the following desired effects: (1) killing a vector or decreasing the lifespan of a vector by greater than about 10%, 20%, 30%, 40%, 50%, 60%, 70%, 80%, 90%, 95%, 99%, or 100%; (2) decreasing a vector population by greater than about 10%, 20%, 30%, 40%, 50%, 60%, 70%, 80%, 90%, 95%, 99%, or 100%; (3) decreasing the growth or body weight or body mass of a vector by greater than about 10%, 20%, 30%, 40%, 50%, 60%, 70%, 80%, 90%, 95%, 99%, or 100%; (4) decreasing the development of a vector by greater than about 10%, 20%, 30%, 40%, 50%, 60%, 70%, 80%, 90%, 95%, 99%, or 100%; (5) decreasing the competence of a vector by greater than about 10%, 20%, 30%, 40%, 50%, 60%, 70%, 80%, 90%, 95%, 99%, or 100%; (6) decreasing the metabolic rate or activity of a vector by greater than about 10%, 20%, 30%, 40%, 50%, 60%, 70%, 80%, 90%, 95%, 99%, or 100%; (7) decreasing the mobility of a vector by greater than about 10%, 20%, 30%, 40%, 50%, 60%, 70%, 80%, 90%, 95%, 99%, or 100%; (8) decreasing the reproductive rate of a vector by greater than about 10%, 20%, 30%, 40%, 50%, 60%, 70%, 80%, 90%, 95%, 99%, or 100%; (9) decreasing pathogen transmission (vertical or horizontal transmission) by a vector by greater than about 10%, 20%, 30%, 40%, 50%, 60%, 70%, 80%, 90%, 95%, 99%, or 100%; and/or (10) increasing susceptibility of a vector to a pesticide(s) by greater than about 10%, 20%, 30%, 40%, 50%, 60%, 70%, 80%, 90%, 95%, 99%, or 100% or more. A decrease in vector fitness can be determined in comparison to an untreated vector.

As used herein, the term “untreated vector” refers to a vector that has not been exposed to, contacted with, and/or delivered the vector control composition, including (i) a separate vector that has not been exposed to, contacted with, and/or delivered the vector control composition, or (ii) the same vector undergoing treatment assessed at a time point prior to exposure to, contact with, and/or delivery of the vector control composition.

Notably, decreasing the fitness of a pathogen to control the spread of the pathogen can be a consequence of delivery of the vector control composition to a vector carrying the pathogen, as a decrease in the fitness of the vector correspondingly decreases the fitness of the pathogen carried by the vector, thereby interrupting the transmission cycle of the pathogen which can reduce the incidence and prevalence of vector-borne diseases. As illustrative examples, decreasing the fitness of a pathogen may manifest as a deterioration or decline in the physiology of the pathogen as a consequence of delivery of the vector control composition to the vector carrying the pathogen. In some instances, the fitness of a pathogen may be measured by one or more parameters, including but not limited to survival, viability, lifespan, health, body weight, metabolic rate or activity, development, mobility, fertility, or reproductive rate in comparison to an untreated pathogen. In some instances, the decrease in pathogen fitness may manifest as an increase in the pathogen's sensitivity to an antipathogen agent and/or a decrease in the pathogen's resistance to an antipathogen agent in comparison to an untreated pathogen. Further, in some instances, the decrease in pathogen fitness may manifest as other fitness disadvantages, such as a decreased tolerance to certain environmental factors (for example, a high or low temperature tolerance), a decreased ability to survive in certain habitats, or a decreased ability to sustain a certain diet in comparison to an untreated pathogen.

The vector control composition of the present invention includes an acaricide which can be effective to decrease the fitness of an acarine vector. As to particular embodiments, the acaricide can be a pyrazole acaricide. As to particular embodiments, the pyrazole acaricide can be a phenylpyrazole acaricide. As to particular embodiments, the phenylpyrazole acaricide can be acetoprole. As to particular embodiments, the phenylpyrazole acaricide can be vaniliprole. As to particular embodiments, the phenylpyrazole acaricide can be fipronil (CAS Number: 120068-37-3), which has a molecular formula of C₁₂H₄C₁₂F₆N₄OS and the structure shown in Formula I.

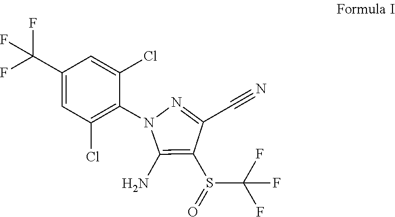

Fipronil is a broad-spectrum acaricide which targets the gamma-aminobutyric acid (GABA) receptor and glutamate-gated chloride channels in the central nervous system of acarines, both of which play a crucial role in regulating nerve impulses. When fipronil binds to these receptors, it blocks the passage of chloride ions through the channels, leading to hyperexcitation of the nervous system, ultimately causing paralysis and death of the acarine.

Mammals, including humans, also have GABA receptors and glutamate-gated chloride channels, but the structure of these receptors differs between acarines and mammals. Fipronil has a higher affinity for acarine receptors compared to mammalian receptors, which means fipronil is more selective in its action on acarine nervous systems and less likely to affect mammals. This selective action on acarine receptors makes fipronil an effective acaricide while minimizing its impact on non-target organisms, including mammals.

As to particular embodiments, the vector control composition of the present invention can include fipronil in an amount effective to decrease the fitness of an acarine vector, whereby this amount can be a relatively low dose of fipronil.

It will be appreciated that minimizing the amount of active agent (such as fipronil) in the vector control composition is advantageous for many reasons, for example (i) because it decreases the amount of active agent introduced into the environment; (ii) because it reduces the risk of non-target organisms ingesting a lethal dose of the active agent; (iii) because it may decrease the overall cost of the vector control composition; and (iv) because it may decrease the risk associated with preparing and handling the vector control composition.

As to particular embodiments, the vector control composition can include a relatively low dose of fipronil which can be effective to decrease the fitness of an acarine vector upon ingestion of the low-dose fipronil by the acarine vector. Correspondingly, the low-dose fipronil can be formulated for oral delivery to the acarine vector. As to particular embodiments, for oral delivery to the acarine vector, the low-dose fipronil can be formulated for ingestion by the acarine vector during blood-feeding on a host.

Additionally, the relatively low dose of fipronil can be effective to decrease the fitness of an acarine vector upon ingestion of the vector control composition by a host. Correspondingly, the vector control composition can be formulated for oral delivery to a host. After a host ingests the vector control composition, at least a portion of the fipronil can be absorbed into the host's bloodstream and thus, can be systemically distributed. Consequently, when blood-feeding on the host, the acarine vector can incidentally ingest fipronil present in the blood of the host.

As to particular embodiments, the vector control composition can include an amount of fipronil selected from the group including or consisting of: not greater than about 0.005% by weight of the composition; less than about 0.005% by weight of the composition; not greater than about 0.0049% by weight of the composition; not greater than about 0.0045% by weight of the composition; not greater than about 0.004% by weight of the composition; not greater than about 0.0035% by weight of the composition; not greater than about 0.003% by weight of the composition; not greater than about 0.0025% by weight of the composition; not greater than about 0.002% by weight of the composition; not greater than about 0.0015% by weight of the composition; and not greater than about 0.001% by weight of the composition. Upon ingestion, this amount of fipronil can be effective to decrease the fitness of an acarine vector.

As to particular embodiments, the vector control composition can include an amount of fipronil selected from the group including or consisting of: a range of between about 0.001% and about 0.005% by weight of the composition; a range of between about 0.001% and less than about 0.005% by weight of the composition; a range of between about 0.001% and about 0.0049% by weight of the composition; a range of between about 0.001% and about 0.0045% by weight of the composition; a range of between about 0.001% and about 0.004% by weight of the composition; a range of between about 0.001% and about 0.0035% by weight of the composition; a range of between about 0.001% and about 0.003% by weight of the composition; a range of between about 0.001% and about 0.0025% by weight of the composition; a range of between about 0.001% and about 0.002% by weight of the composition; and a range of between about 0.001% and about 0.0015% by weight of the composition. Upon ingestion, this amount of fipronil can be effective to decrease the fitness of an acarine vector.

As but one illustrative example, the vector control composition can include about 0.0025% fipronil by weight of the composition. Upon ingestion, this amount of fipronil can be effective to decrease the fitness of an acarine vector.

As but a second illustrative example, the vector control composition can include about 0.001% fipronil by weight of the composition. Upon ingestion, this amount of fipronil can be effective to decrease the fitness of an acarine vector.

In addition to effectively decreasing the fitness of a vector, as to particular embodiments, the present vector control composition including a relatively low dose of fipronil can further be effective to prevent an infection (the presence or colonization of a pathogen in, on, or around an organism) in an organism at risk of such an infection with a pathogen, particularly when the infection can decrease the fitness of the organism, e.g., by causing disease, disease symptoms, or an immune/inflammatory response. As used herein, the term “prevent an infection” refers to preventing the onset of an infection and/or symptoms or conditions associated with an infection.

### Delivery

A vector can be exposed to the active agent of the vector control composition described herein in any suitable manner that permits delivering the active agent to the vector. Following, the method of decreasing the fitness of a vector capable of transmitting a pathogen includes delivering the active agent to the vector.

As stated above, as to particular embodiments, the vector control composition can be formulated for oral delivery to a host of the vector. To reiterate, after the host ingests the vector control composition, at least a portion of the active agent can be absorbed into the host's bloodstream and thus, can be systemically distributed; when blood-feeding on the host, the vector can incidentally ingest the active agent present in the blood of the host.

As to particular embodiments, for oral delivery to a host, the vector control composition can be formulated as a comestible composition for ingestion by the host. As to particular embodiments, the vector control composition can be formulated as comestible matter, such as feed (or food or diet), which can be ingested by a host; such a formulation can be relatively easy to deliver and may increase uptake of the vector control composition by the host. In addition, such a formulation can be particularly suited for chronic dosing resulting from multiple feedings, which may be in contrast to acute dosing.

As used herein, the term “feed” refers to the combination of at least a purposely selected amount of an active agent and a purposely selected carrier for use as a targeted vector control composition, which is to be contrasted with, for instance, a naturally occurring material. The feed should be both palatable to a target host and edible by the target host.

As to particular embodiments, the feed can comprise at least an amount of a low dose of an acaricide and an acceptable carrier (or excipient), such as a pharmaceutically acceptable carrier suitable for administration to an animal and of course, nontoxic to the animal in the amount employed. The carrier should be palatable to a target host and thus, can comprise any of a numerous and wide variety of edibles, a list of which is herein contemplated but too extensive to include.

As but one illustrative, non-limiting example, the carrier can comprise corn or a corn formulation. For example, the carrier can comprise cornmeal or a cornmeal formulation. Cornmeal, made from ground corn kernels, is primarily composed of carbohydrates, mainly in the form of starch. Additionally, cornmeal can contain protein, fiber, vitamins (such as B vitamins including thiamine (vitamin B1), niacin (vitamin B3), and folate (vitamin B9), minerals (such as magnesium, phosphorus, and potassium), fat (primarily in the form of unsaturated fats), and antioxidants (such as carotenoids).

As to particular embodiments, the feed can primarily comprise or consist essentially of or consist of corn. As to particular embodiments, the feed can comprise or consist essentially of or consist of not less than 95% corn. As to particular embodiments, the feed can comprise or consist essentially of or consist of not less than 95.5% corn. As to particular embodiments, the feed can comprise or consist essentially of or consist of not less than 96% corn. As to particular embodiments, the feed can comprise or consist essentially of or consist of not less than 96.5% corn. As to particular embodiments, the feed can comprise or consist essentially of or consist of not less than 97% corn. As to particular embodiments, the feed can comprise or consist essentially of or consist of not less than 97.5% corn. As to particular embodiments, the feed can comprise or consist essentially of or consist of not less than 98% corn. As to particular embodiments, the feed can comprise or consist essentially of or consist of not less than 98.5% corn. As to particular embodiments, the feed can comprise or consist essentially of or consist of not less than 99% corn. As to particular embodiments, the feed can comprise or consist essentially of or consist of not less than 99.5% corn. Of course, for all of the above embodiments, the feed also includes the active agent.

As but a second illustrative, non-limiting example, the carrier can comprise sugar beets or a sugar beet formulation. Sugar beets are primarily valued for their high sugar content (predominantly sucrose), which typically ranges from 15% to 20% of their total weight. Also, sugar beets can contain fiber, various vitamins and minerals (such as vitamin C, folate, potassium, magnesium, and iron), nitrate, betaine, phytonutrients (such as betalains which are responsible for their characteristic red color), and protein.

As to particular embodiments, the feed can also include one or more attractants, such as a flavorant, a palatant, an odorant, or the like, for attracting a host. The attractant may be embodied by the carrier or can be discrete from the carrier, depending upon the formulation. As illustrative, non-limiting examples, for a herbivorous target host, the attractant can be a plant-derived component, such as corn, corn products, corn meal, wheat, wheat products, oats, oat products, cereals, sugars, syrups, fruits, vegetables, seeds, nuts, or the like.

As to particular embodiments, the carrier, the attractant, or both, can be selected with consideration for non-target organisms to reduce potential uptake of the feed by the non-target organisms. Accordingly, the selection of the particular type and amount of carrier, attractant, or both, may vary depending upon the non-target organisms desired to deter. Furthermore, as to particular embodiments, the feed can also include one or more repellants intended to repel the non-target organisms.

As to particular embodiments, the feed can, but need not necessarily, further include a colorant or dye which can visibly color the feed, thereby allowing visible identification of feed including the colorant.

The feed can, but need not necessarily, include more than one carrier and attractant, and can further include additional components, such as additives, preservatives, plasticizers, humectants, buffering agents, oils, or the like.

As to particular embodiments, the vector control composition formulated as feed can be in a substantially solid form, meaning firm and stable in shape (not fluid or liquid). As illustrative, nonlimiting examples, the feed can be in the form of a powder, granules, pellets, briquettes, bricks, blocks, or the like. It will be appreciated that the actual shape of the feed may not be a crucial parameter, and that any obtainable shape is within the scope of the present invention.

As to particular embodiments, the vector control composition formulated as feed can further be substantially homogenous or homogenous, meaning of uniform composition throughout.

The vector control composition formulated as feed may be an immediate-release formulation, an extended release formulation, or a delayed-release formulation, depending upon the embodiment.

To provide the vector control composition formulated as feed, the active agent can be combined with a carrier. For example, an amount of a low dose of an acaricide can be incorporated into the carrier or dispersed throughout the carrier, which may be accomplished by combining or mixing the desired amount of a low dose of an acaricide and the desired amount of a carrier.

Amounts and locations for disposition of the vector control composition described herein can be generally determined at least by the habits of the vector and/or the host as well as the physical and functional characteristics of the vector control composition.

### Example 1

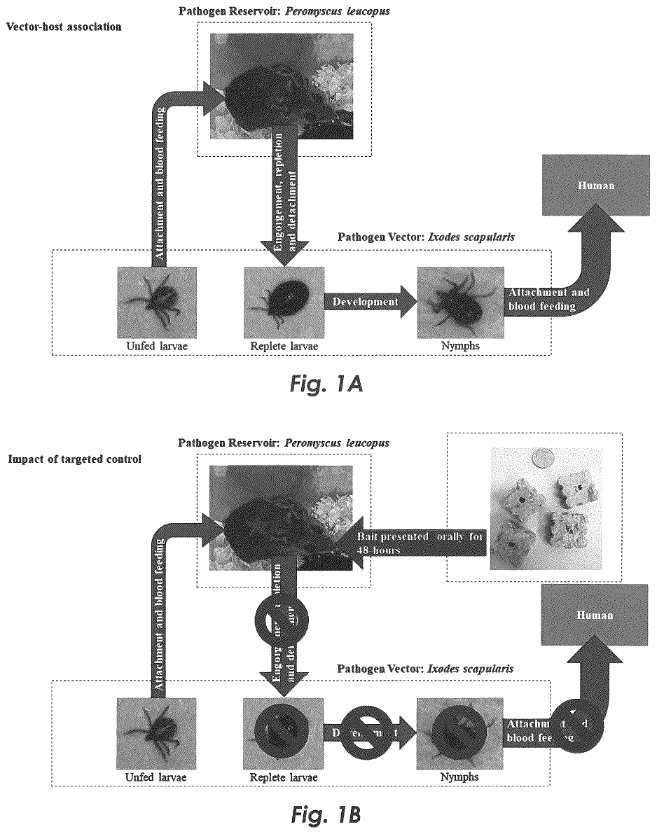

A laboratory study was conducted to determine the efficacy of inventive low dose fipronil compositions presented to white-footed mice (Peromyscus leucopus) in controlling blacklegged tick larvae (Ixodes scapularis). The vector-host association and treatment concept are presented in FIGS. 1A and 1B, respectively.

### Low Dose Fipronil Compositions

The efficacy of two low dose fipronil compositions was determined, whereby both compositions comprised a solid feed formulation and in particular, a corn-based pelleted feed formulation. The first low dose fipronil composition comprised a nominal fipronil concentration of 0.001% and is hereinafter termed “Composition I.” The second low dose fipronil composition comprised a nominal fipronil concentration of 0.0025% and is hereinafter termed “Composition II.”

### Study Groups

Mice were randomly assigned to one of four treatment groups or three control groups, with each group having six mice total (three males and three females, all sexually mature young adults). Mice in the treatment groups were exposed to Composition I or Composition II as detailed below.

On Day 1 post exposure to Composition I, tick larvae were allowed to attach to mice in Treatment Group 1 (TG-1).

On Day 8 post exposure to Composition I, tick larvae were allowed to attach to mice in Treatment Group 2 (TG-2).

On Day 15 post exposure to Composition I, tick larvae were allowed to attach to mice in Treatment Group 3 (TG-3).

On Day 15 post exposure to Composition II, tick larvae were allowed to attach to mice in Treatment Group 4 (TG-4).

During the study, mice were housed individually.

### Pre Exposure (Acclimation)

Mice were acclimated to test conditions for four days prior to exposure to Composition I or Composition II. During acclimation, mice were presented with commercial rodent diet (Teklad 8604 Rodent Diet; Inotiv, West Lafayette, IN, USA) ad libitum.

### Exposure

At the end of acclimation, each mouse within the Treatment Groups was presented with 30 grams of Composition I or Composition II in a steel feed cup and was exposed thereto for 48 hours. Each mouse within the control groups was presented with the commercial rodent diet which was provided ad libitum. At the end of the exposure period (post exposure), Composition I or Composition II was removed from the Treatment Groups and replaced with the commercial rodent diet provided ad libitum for the remainder of the study. The control groups continued with the commercial rodent diet provided ad libitum.

### Post Exposure Tick Larvae Attachment

At the end of the exposure period, tick larvae were allowed to attach to each mouse in the Treatment Groups on (i) Day 1 post exposure to Composition I (TG-1), (ii) Day 8 post exposure to Composition I (TG-2), (iii) Day 15 post exposure to Composition I (TG-3), or (iv) Day 15 post exposure to Composition II (TG-4). Tick larvae were also allowed to attach to each mouse in the control groups on (i) Day 1 to correspond to TG-1 for Control Group 1 (CG-1), (ii) Day 8 to correspond to TG-2 for Control Group 2 (CG-2), and (iii) Day 15 to correspond to TG-3 and TG-4 for Control Group 3 (CG-3).

For tick larvae attachment, a patch of fur between each mouse's shoulder blades was shaved to make the skin easily accessible to the tick larvae. A small plastic capsule was secured to the skin using an adhesive including colophony and beeswax. About 40 tick larvae were transferred into each capsule. Following, the top of each capsule was secured with a plastic lid. Tick larvae feeding to repletion and detaching from mice were maintained in a desiccator at high humidity (~90%) and monitored routinely for eight weeks following detachment to determine if they were able to successfully develop into nymphs. At the end of the attachment period, plasma was collected from all mice for analyses.

### Composition Consumption

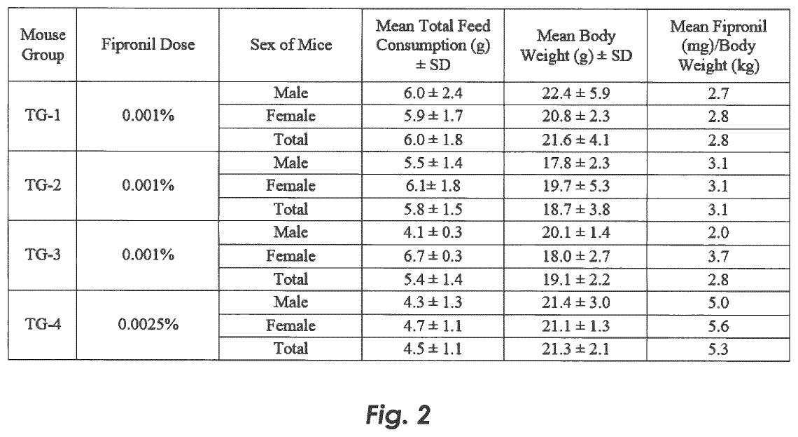

The amount of Composition I or Composition II consumed by mice in the Treatment Groups is presented in FIG. 2. Consumption averaged (i) 6.0 grams (g) for mice in TG-1, (ii) 5.8 g for mice in TG-2, (iii) 5.4 g for mice in TG-3, and (iv) 4.5 g for mice in TG-4. Correspondingly, the mean milligrams (mg) of fipronil per kilogram (kg) of body weight was (i) 2.8 mg/kg for mice in TG-1, (ii) 3.1 mg/kg for mice in TG-2, (iii) 2.8 mg/kg for mice in TG-3, and (iv) 5.3 mg/kg for mice in TG-4. No mice were observed to have any adverse symptoms from consumption of Composition I or Composition II.

### Tick Larvae Observations and Efficacy

Tick larvae were observed and counted on Day 2 post tick larvae attachment, Day 3 post tick larvae attachment, and Day 4 post tick larvae attachment.

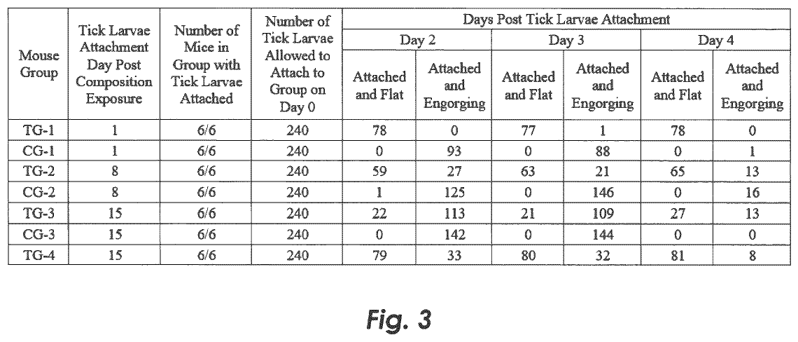

Tick larvae attachment status and feeding status (engorging or flat (not engorging)) are presented in FIG. 3. For attached tick larvae, the number of tick larvae engorging on Day 2 and Day 3 post tick larvae attachment was significantly greater within the Control Groups relative to the Treatment Groups (Student's t, t_{(81.771)}=−7.576, p<0.0001). Conversely, the number of flat tick larvae was significantly greater within the Treatment Groups relative to the Control Groups (Student's t, t_{(47.072)}=9.889, p<0.0001).

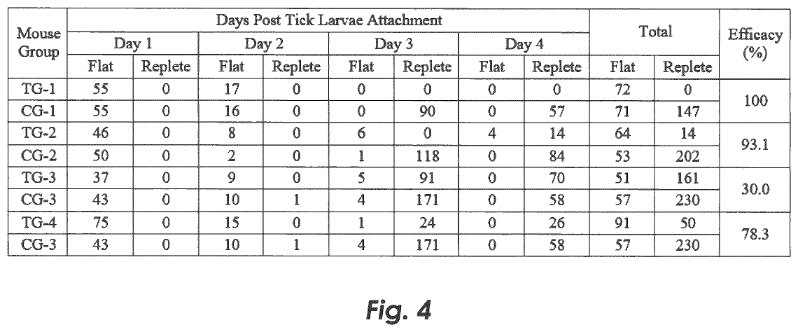

Tick larvae detached status (replete or flat (not engorged)) are presented in FIG. 4. For detached tick larvae, the number of tick larvae feeding to repletion and detaching was significantly greater within the Control Groups relative to the Treatment Groups (Student's t, t_{(66.111)}=−5.175, p<0.0001). A total of 147 replete tick larvae were collected from CG-1 relative to 0 replete tick larvae collected from TG-1. A total of 202 replete tick larvae were collected from CG-2 relative to 14 replete tick larvae from TG-2. A total of 229 replete tick larvae were collected from CG-3 relative to 161 replete tick larvae from TG-3. A total of 229 replete tick larvae were collected from CG-3 relative to 50 replete tick larvae from TG-4.

Efficacy of preventing tick larvae parasitizing mice from feeding to repletion post exposure to low dose fipronil compositions is also presented in FIG. 4, whereby efficacy was calculated using Abbott's formula:

$$Efficacy ( \% ) = 100 \times ( \frac{CG - TG}{CG} )$$

Following, efficacy of preventing tick larvae parasitizing mice from feeding to repletion on Day 1 post exposure to Composition I was 100%. Efficacy of preventing tick larvae parasitizing mice from feeding to repletion on Day 8 post exposure to Composition I was 93.1%. Efficacy of preventing tick larvae parasitizing mice from feeding to repletion on Day 15 post exposure to Composition I was 30%. Efficacy of preventing tick larvae parasitizing mice from feeding to repletion on Day 15 post exposure to Composition II was 78.2%.

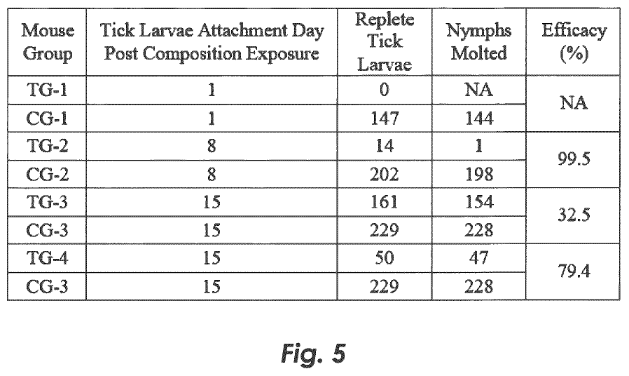

Tick larvae molting success (nymphal development) is presented in FIG. 5. Zero replete tick larvae were collected from TG-1 and thus, no molting was recorded (100% efficacy). Of 147 replete tick larvae collected from CG-1, 144 (98%) molted. Of 14 replete tick larvae collected from TG-2, 1 (7%) molted. Of 202 replete tick larvae collected from CG-2, 198 (98%) molted. Of 229 replete tick larvae collected from CG-3, 228 (99.6%) molted. Of 161 replete tick larvae collected from TG-3, 154 (95.7%) molted. Of 50 replete tick larvae collected from TG-4, 47 (94%) molted.

Efficacy of preventing tick larvae parasitizing mice from reaching the nymphal stage post exposure to low dose fipronil compositions is also presented in FIG. 5, whereby efficacy was calculated using Abbott's formula:

$$Efficacy ( \% ) = 100 \times ( \frac{CG - TG}{CG} )$$

Following, efficacy of preventing tick larvae parasitizing mice from reaching the nymphal stage on Day 1 post exposure to Composition I was 100%. Efficacy of preventing tick larvae parasitizing mice from reaching the nymphal stage on Day 8 post exposure to Composition I was 99.5%. Efficacy of preventing tick larvae parasitizing mice from reaching the nymphal stage on Day 15 post exposure to Composition I was 32.5%. Efficacy of preventing tick larvae parasitizing mice from reaching the nymphal stage on Day 15 post exposure to Composition II was 79.4%.

### Fipronil and Fipronil Sulfone Plasma Concentrations

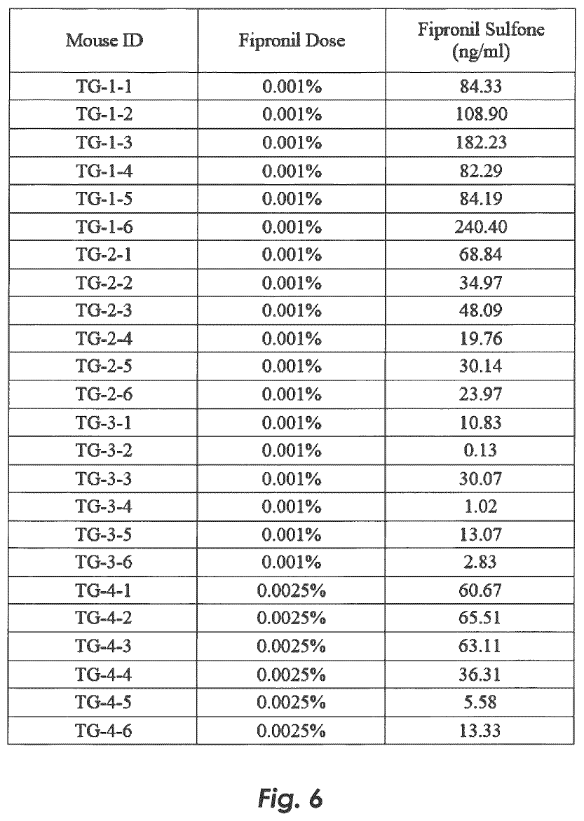

No fipronil was detected in any plasma samples from mice in Treatment Groups. Concentrations of fipronil sulfone (a metabolite of fipronil) in plasma samples from mice in Treatment Groups are presented in FIG. 6. The average fipronil sulfone concentration in plasma collected from TG-1 mice on Day 5 post exposure to Composition I was 130.4±66.1 nanograms/milliliter (ng/ml). The average fipronil sulfone concentration in plasma collected from TG-2 mice on Day 12 post exposure to Composition I was 37.6±18.2 ng/ml. Of note, 11 of the 14 replete tick larvae were collected from the mouse with the lowest fipronil sulfone plasma level within TG-2 (19.8 ng/ml). The average fipronil sulfone concentration in plasma collected from TG-3 mice on Day 19 post exposure to Composition I was 9.7±11.3 ng/ml. Of note, the only mouse to have no tick larvae feed to full repletion and detach had the highest fipronil sulfone plasma level within TG-3 (30.1 ng/ml). The average fipronil sulfone concentration in plasma collected from TG-4 mice on Day 19 post exposure to Composition II was 40.8±26.5 ng/ml. Of note, substantially more replete tick larvae were collected from TG-4-5 and TG-4-6 (n=49) relative to the other four TG-4 mice, which correlates with the marginal fipronil sulfone concentrations in plasma from TG-4-5 (5.6 ng/ml) and TG-4-6 (13.3 ng/ml) relative to the other four TG-4 mice.

### Example 2

A laboratory study was conducted to determine the efficacy of an inventive low dose fipronil composition presented to white-footed mice (Peromyscus leucopus) in controlling blacklegged tick larvae (Ixodes scapularis). The vector-host association and treatment concept are presented in FIGS. 1A and 1B, respectively.

### Low Dose Fipronil Compositions

The efficacy of a low dose fipronil composition was determined, whereby the composition comprised a solid feed formulation and in particular, a sugar beet-based pelleted feed formulation. The low dose fipronil composition comprised a nominal fipronil concentration of 0.0025% and is hereinafter termed “Composition III.”

### Study Groups

Mice were randomly assigned to one of two treatment groups or two control groups, with each group having six mice total (three males and three females, all sexually mature young adults).

On Day 1 post exposure to Composition III, tick larvae were allowed to attach to mice in Treatment Group 1 (TG-1).

On Day 8 post exposure to Composition III, tick larvae were allowed to attach to mice in Treatment Group 2 (TG-2).

During the study, mice were housed individually.

### Pre Exposure (Acclimation)

Mice were acclimated to test conditions for two days prior to exposure to Composition III. During acclimation, mice were presented with commercial rodent diet (Teklad 8604 Rodent Diet; Inotiv, West Lafayette, IN, USA) ad libitum.

### Exposure

At the end of acclimation, each mouse within the Treatment Groups was presented with 30 grams of Composition III in a steel feed cup and was exposed thereto for 48 hours. Each mouse within the control groups was presented with the commercial rodent diet which was provided ad libitum. At the end of the exposure period (post exposure), Composition III was removed from the Treatment Groups and replaced with the commercial rodent diet provided ad libitum for the remainder of the study. The control groups continued with the commercial rodent diet provided ad libitum.

### Post Exposure Tick Larvae Attachment

At the end of the exposure period, tick larvae were allowed to attach to each mouse in the Treatment Groups on (i) Day 1 post exposure to Composition III (TG-1), or (ii) Day 8 post exposure to Composition III (TG-2). Tick larvae were also allowed to attach to each mouse in the control groups on (i) Day 1 to correspond to TG-1 for Control Group 1 (CG-1), or (ii) Day 8 to correspond to TG-2 for Control Group 2 (CG-2).

For tick larvae attachment, a patch of fur between each mouse's shoulder blades was shaved to make the skin easily accessible to the tick larvae. A small plastic capsule was secured to the skin using an adhesive including colophony and beeswax. About 40 tick larvae were transferred into each capsule. Following, the top of each capsule was secured with a plastic lid. Tick larvae feeding to repletion and detaching from mice were maintained in a desiccator at high humidity (~90%) and monitored routinely for eight weeks following detachment to determine if they were able to successfully develop into nymphs. At the end of the attachment period, plasma was collected from all mice for analyses.

### Composition Consumption

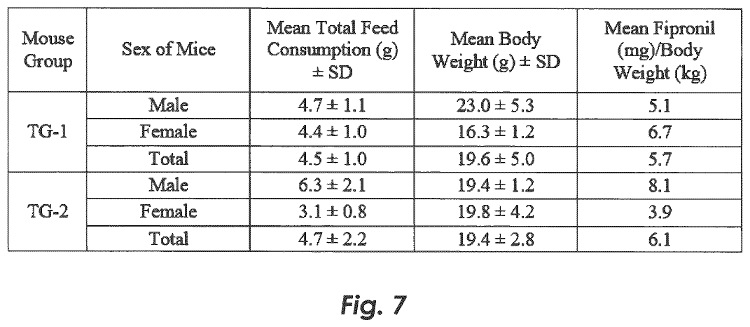

The amount of Composition III consumed by mice in the Treatment Groups is presented in FIG. 7. Consumption averaged (i) 4.5 grams (g) for mice in TG-1, and (ii) 4.7 g for mice in TG-2. Correspondingly, the mean milligrams (mg) of fipronil per kilogram (kg) of body weight was (i) 5.7 mg/kg for mice in TG-1, and (ii) 6.1 mg/kg for mice in TG-2. No mice were observed to have any adverse symptoms from consumption of Composition III.

### Tick Larvae Observations and Efficacy

Tick larvae were observed and counted on Day 2 post tick larvae attachment, Day 3 post tick larvae attachment, and Day 4 post tick larvae attachment.

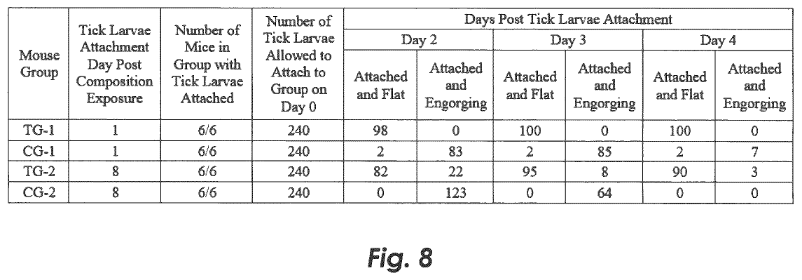

Tick larvae attachment status and feeding status (engorging or flat (not engorging)) are presented in FIG. 8. For attached tick larvae, the number of tick larvae engorging on Day 2 and Day 3 post tick larvae attachment was significantly greater within the Control Groups relative to the Treatment Groups (Student's t, t_{(38.302)}=−7.720, p<0.0001). Conversely, the number of flat tick larvae was significantly greater within the Treatment Groups relative to the Control Groups (Student's t, t_{(23.544)}=14.505, p<0.0001).

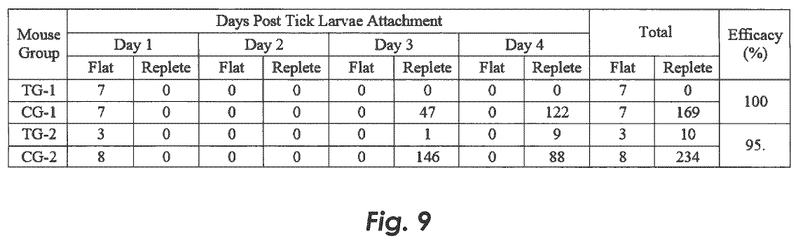

Tick larvae detached status (replete or flat (not engorged)) are presented in FIG. 9. For detached tick larvae, the number of tick larvae feeding to repletion and detaching was significantly greater within the Control Groups relative to the Treatment Groups (Student's t, t_{(24.601)}=−8.002, p<0.0001). A total of 169 replete tick larvae were collected from CG-1 relative to 0 replete tick larvae collected from TG-1. A total of 234 replete tick larvae were collected from CG-2 relative to 10 replete tick larvae from TG-2.

Efficacy of preventing tick larvae parasitizing mice from feeding to repletion post exposure to a low dose fipronil composition is also presented in FIG. 9, whereby efficacy was calculated using Abbott's formula:

$$Efficacy ( \% ) = 100 \times ( \frac{CG - TG}{CG} )$$

Following, efficacy of preventing tick larvae parasitizing mice from feeding to repletion on Day 1 post exposure to Composition III was 100%. Efficacy of preventing tick larvae parasitizing mice from feeding to repletion on Day 8 post exposure to Composition III was 95.7%.

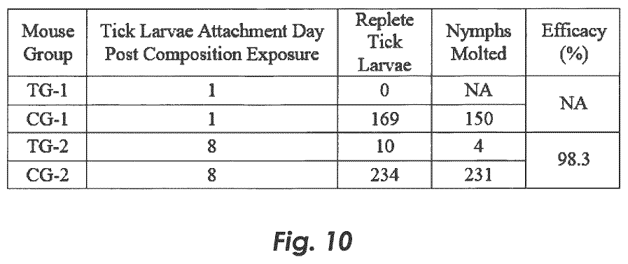

Tick larvae molting success (nymphal development) is presented in FIG. 10. Zero replete tick larvae were collected from TG-1 and thus, no molting was recorded (100% efficacy). Of 169 replete tick larvae collected from CG-1, 150 (88.8%) molted. Of 10 replete tick larvae collected from TG-2, 4 (40%) molted. Of 234 replete tick larvae collected from CG-2, 231 (98.7%) molted.

Efficacy of preventing tick larvae parasitizing mice from reaching the nymphal stage post exposure to a low dose fipronil composition is also presented in FIG. 10, whereby efficacy was calculated using Abbott's formula:

$$Efficacy ( \% ) = 100 \times ( \frac{CG - TG}{CG} )$$

Following, efficacy of preventing tick larvae parasitizing mice from reaching the nymphal stage on Day 1 post exposure to Composition III was 100%. Efficacy of preventing tick larvae parasitizing mice from reaching the nymphal stage on Day 8 post exposure to Composition III was 98.3%.

### Fipronil and Fipronil Sulfone Plasma Concentrations

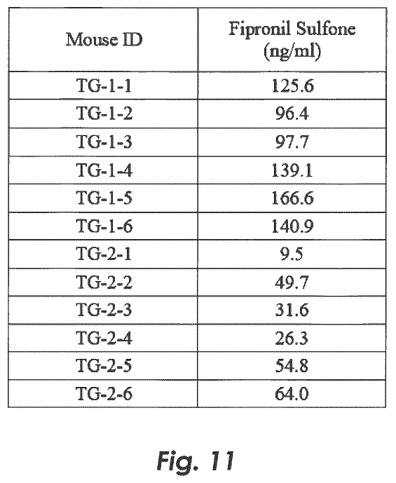

No fipronil was detected in any plasma samples from mice in Treatment Groups. Concentrations of fipronil sulfone (a metabolite of fipronil) in plasma samples from mice in Treatment Groups are presented in FIG. 11. The average fipronil sulfone concentration in plasma collected from TG-1 mice on Day 5 post exposure to Composition III was 127.7±27.2 nanograms/milliliter (ng/ml). The average fipronil sulfone concentration in plasma collected from TG-2 mice on Day 12 post exposure to Composition III was 39.3±20.4 ng/ml. Of note, only one mouse within both Treatment Groups had tick larvae which fed to repletion and detached; this mouse (TG-2-1) had the lowest fipronil sulfone plasma level within both Treatment Groups (9.5 ng/ml).

As can be easily understood from the foregoing, the basic concepts of the present invention may be embodied in a variety of ways. The invention involves numerous and varied embodiments of a vector control composition and methods for using the same.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “formulation” should be understood to encompass disclosure of the act of “formulating”—whether explicitly discussed or not—and, conversely, were there effectively disclosure of the act of “formulating,” such a disclosure should be understood to encompass disclosure of a “formulation” and even a “means for formulating.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the vector control compositions and methods for using the same herein disclosed and described, ii) the related methods disclosed and described, iii) similar, equivalent, and even implicit variations of each of these compositions and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice versa as necessary to define the matter for which protection is sought by this application or by any subsequent application or continuation, division, or continuation in part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation in part application thereof or any reissue or extension thereon.

Additionally, the claims set forth in this specification are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A vector control composition, comprising:
an active agent effective to decrease the fitness of a vector capable of transmitting a pathogen;
wherein said active agent comprises a low dose of an acaricide; and
wherein said active agent is formulated for oral delivery.

2. The composition of claim 1, wherein said vector comprises an acarine.

3. The composition of claim 2, wherein said acarine comprises a parasitic acarine.

4. The composition of claim 3, wherein said parasitic acarine comprises a tick.

5. The composition of claim 4, wherein said tick comprises Ixodes scapularis.

6. The composition of claim 4, wherein said tick comprises a blacklegged tick.

7. The composition of claim 1, wherein said pathogen comprises a bacterium.

8. The composition of claim 1, wherein said pathogen comprises a virus.

9. The composition of claim 1, wherein said pathogen comprises a parasite.

10. The composition of claim 1, wherein said pathogen comprises a protozoon.

11. The composition of claim 1, wherein said pathogen is the causative agent of anaplasmosis.

12. The composition of claim 1, wherein said pathogen is the causative agent of babesiosis.

13. The composition of claim 1, wherein said pathogen is the causative agent of ehrlichiosis.

14. The composition of claim 1, wherein said pathogen is the causative agent of Lyme disease.

15. The composition of claim 1, wherein said low dose of said acaricide is delivered to said vector via a host of said vector.

16. The composition of claim 15, wherein said host comprises a mammal.

17. The composition of claim 15, wherein said host comprises a rodent.

18. The composition of claim 17, wherein said host comprises a mouse.

19. The composition of claim 1, wherein said acaricide comprises a pyrazole acaricide.

20. The composition of claim 19, wherein said pyrazole acaricide comprises a phenylpyrazole acaricide.

21. The composition of claim 20, wherein said phenylpyrazole acaricide comprises acetoprole.

22. The composition of claim 20, wherein said phenylpyrazole acaricide comprises vaniliprole.

23. The composition of claim 20, wherein said phenylpyrazole acaricide comprises fipronil.

24. The composition of claim 23, wherein said low dose of said fipronil is effective to decrease said fitness of said vector upon ingestion.

25. The composition of claim 24, wherein said low dose of said fipronil is formulated for oral delivery to said vector.

26. The composition of claim 25, wherein said low dose of said fipronil is formulated for ingestion by said vector during blood-feeding on a host.

27. The composition of claim 26, wherein said low dose of said fipronil is effective to decrease said fitness of said vector upon ingestion of said composition by said host.

28. The composition of claim 27, wherein said composition is formulated for oral delivery to said host.

29. The composition of claim 24, wherein an amount of said low dose of said fipronil is not greater than about 0.005% by weight of said composition.

30. The composition of claim 24, wherein an amount of said low dose of said fipronil is less than about 0.005% by weight of said composition.

31. The composition of claim 24, wherein an amount of said low dose of said fipronil is not greater than about 0.0049% by weight of said composition.

32. The composition of claim 24, wherein an amount of said low dose of said fipronil is not greater than about 0.0045% by weight of said composition.

33. The composition of claim 24, wherein an amount of said low dose of said fipronil is not greater than about 0.004% by weight of said composition.

34. The composition of claim 24, wherein an amount of said low dose of said fipronil is not greater than about 0.0035% by weight of said composition.

35. The composition of claim 24, wherein an amount of said low dose of said fipronil is not greater than about 0.003% by weight of said composition.

36. The composition of claim 24, wherein an amount of said low dose of said fipronil is not greater than about 0.0025% by weight of said composition.

37. The composition of claim 24, wherein an amount of said low dose of said fipronil is not greater than about 0.002% by weight of said composition.

38. The composition of claim 24, wherein an amount of said low dose of said fipronil is not greater than about 0.0015% by weight of said composition.

39. The composition of claim 24, wherein an amount of said low dose of said fipronil is not greater than about 0.001% by weight of said composition.

40. The composition of claim 24, wherein an amount of said low dose of said fipronil is in a range of between about 0.001% and about 0.005% by weight of said composition.

41. The composition of claim 24, wherein an amount of said low dose of said fipronil is in a range of between about 0.001% and less than about 0.005% by weight of said composition.

42. The composition of claim 24, wherein an amount of said low dose of said fipronil is in a range of between about 0.001% and about 0.0049% by weight of said composition.

43. The composition of claim 24, wherein an amount of said low dose of said fipronil is in a range of between about 0.001% and about 0.0045% by weight of said composition.

44. The composition of claim 24, wherein an amount of said low dose of said fipronil is in a range of between about 0.001% and about 0.004% by weight of said composition.

45. The composition of claim 24, wherein an amount of said low dose of said fipronil is in a range of between about 0.001% and about 0.0035% by weight of said composition.

46. The composition of claim 24, wherein an amount of said low dose of said fipronil is in a range of between about 0.001% and about 0.003% by weight of said composition.

47. The composition of claim 24, wherein an amount of said low dose of said fipronil is in a range of between about 0.001% and about 0.0025% by weight of said composition.

48. The composition of claim 24, wherein an amount of said low dose of said fipronil is in a range of between about 0.001% and about 0.002% by weight of said composition.

49. The composition of claim 24, wherein an amount of said low dose of said fipronil is in a range of between about 0.001% and about 0.0015% by weight of said composition.

50. The composition of claim 24, comprising about 0.001% of said fipronil by weight of said composition.

51. The composition of claim 15, wherein said composition is formulated as a comestible composition for ingestion by said host.

52. The composition of claim 51, wherein said composition is formulated as feed for said host.

53. The composition of claim 52, further comprising a carrier palatable to a target said host and edible by said target host.

54. The composition of claim 53, wherein said carrier comprises corn.

55. The composition of claim 53, wherein said carrier comprises cornmeal.

56. The composition of claim 53, further comprising an attractant which attracts said target host.

57. The composition of claim 52, wherein said feed comprises a substantially solid form.

58. The composition of claim 1, wherein said composition controls ticks parasitizing white-footed mice.

59. A vector control composition, comprising:
an active agent effective to decrease the fitness of a vector capable of transmitting a pathogen;
wherein said active agent comprises an acaricide; and
wherein said active agent is formulated for oral delivery.

60. The composition of claim 59, wherein said active agent consists of said acaricide.

61. The composition of claim 59, wherein said active agent comprises a low dose of said acaricide.

62. The composition of claim 59, wherein said active agent consists of a low dose of said acaricide.

63. The composition of claim 59, wherein said active agent comprises fipronil.

64. The composition of claim 59, wherein said active agent consists of fipronil.

65. The composition of claim 59, wherein said active agent comprises a low dose of fipronil.

66. The composition of claim 59, wherein said active agent consists of a low dose of fipronil.

67. The composition of claim 59, further comprising one or more additional active agents.

68. A vector control composition, comprising:
an active agent effective to decrease the fitness of a vector capable of transmitting a pathogen;
wherein said active agent comprises an acaricide;
wherein said active agent is formulated for oral delivery to a host of said vector; and
wherein said host comprises a rodent.

69. A vector control composition, comprising:
an active agent effective to decrease the fitness of a vector capable of transmitting a pathogen;
wherein said active agent comprises a low dose of an acaricide;
wherein said active agent is formulated for oral delivery to a host of said vector; and
wherein said host comprises a rodent.

70. A vector control composition, comprising:
an active agent effective to decrease the fitness of a vector capable of transmitting a pathogen;
wherein said active agent comprises fipronil;
wherein said active agent is formulated for oral delivery to a host of said vector; and
wherein said host comprises a rodent.

71. A vector control composition, comprising:
an active agent effective to decrease the fitness of a vector capable of transmitting a pathogen;
wherein said active agent comprises a low dose of fipronil;
wherein said active agent is formulated for oral delivery to a host of said vector; and
wherein said host comprises a rodent.

72. A vector control composition, comprising:
an active agent effective to decrease the fitness of a vector capable of transmitting a pathogen;
wherein said active agent comprises an acaricide;
wherein said active agent is formulated for oral delivery to a host of said vector; and
wherein said host comprises a mouse.

73. A vector control composition, comprising:
an active agent effective to decrease the fitness of a vector capable of transmitting a pathogen;
wherein said active agent comprises a low dose of an acaricide;
wherein said active agent is formulated for oral delivery to a host of said vector; and
wherein said host comprises a mouse.

74. A vector control composition, comprising:
an active agent effective to decrease the fitness of a vector capable of transmitting a pathogen;
wherein said active agent comprises fipronil;
wherein said active agent is formulated for oral delivery to a host of said vector; and
wherein said host comprises a mouse.

75. A vector control composition, comprising:
an active agent effective to decrease the fitness of a vector capable of transmitting a pathogen;
wherein said active agent comprises a low dose of fipronil;
wherein said active agent is formulated for oral delivery to a host of said vector; and
wherein said host comprises a mouse.

76. A vector control composition, comprising:
an active agent effective to decrease the fitness of a vector capable of transmitting a pathogen;
wherein said active agent comprises an acaricide;
wherein said active agent is formulated for oral delivery to a host of said vector; and
wherein said host comprises a white-footed mouse.

77. A vector control composition, comprising:
an active agent effective to decrease the fitness of a vector capable of transmitting a pathogen;
wherein said active agent comprises a low dose of an acaricide;
wherein said active agent is formulated for oral delivery to a host of said vector; and
wherein said host comprises a white-footed mouse.

78. A vector control composition, comprising:
an active agent effective to decrease the fitness of a vector capable of transmitting a pathogen;
wherein said active agent comprises fipronil;
wherein said active agent is formulated for oral delivery to a host of said vector; and
wherein said host comprises a white-footed mouse.

79. A vector control composition, comprising:
an active agent effective to decrease the fitness of a vector capable of transmitting a pathogen;
wherein said active agent comprises a low dose of fipronil;
wherein said active agent is formulated for oral delivery to a host of said vector; and
wherein said host comprises a white-footed mouse.

80. A method of decreasing the fitness of a vector capable of transmitting a pathogen, comprising:
delivering to a host of said vector an effective amount of a vector control composition of any one of claims 1-79;
wherein said method decreases said fitness of said vector relative to an untreated vector.

81. A method of decreasing the fitness of a vector capable of transmitting a pathogen, comprising:
delivering to a host of said vector an effective amount of a vector control composition comprising:
an active agent effective to decrease said fitness of said vector;
wherein said active agent comprises a low dose of an acaricide; and
wherein said active agent is formulated for oral delivery;
wherein said method decreases said fitness of said vector relative to an untreated vector.

82. The method of claim 81, further comprising orally delivering said vector control composition to said host.

83. The method of claim 81, wherein said vector control composition is formulated as feed for said host.

84. A method of preventing an infection by a pathogen in an animal at risk thereof, comprising:
delivering to a host of a vector of said pathogen an effective amount of a vector control composition comprising:
an active agent effective to decrease said fitness of said vector;
wherein said active agent comprises a low dose of an acaricide; and
wherein said active agent is formulated for oral delivery;
wherein said method decreases the likelihood of said infection in said animal relative to an untreated animal.

85. The method of claim 84, further comprising orally delivering said vector control composition to said host.

86. The method of claim 84, wherein said vector control composition is formulated as feed for said host.
